(19) United States
(12) Patent Application Publication
(10) Pub. No.: US 2004/0136921 A1
(43) Pub. Date: Jul. 15, 2004

(54) Product containing a clear hair lotion sprayable as an aerosol spray

(76) Inventors: Thomas Schulz, Darmstadt (DE); Harald Wendel, Ober-Ramstadt (DE); Susanne Birkel, Darmstadt (DE)

Correspondence Address: STRIKER, STRIKER & STENBY, 103 East Neck Road, Huntington, NY, 11743, US

(21) Appl. No.: 10/732,006

(22) Filed: Dec. 9, 2003

(30) Foreign Application Priority Data
Dec. 11, 2002 (DE) 102 57 858.3

(51) Int. Cl. A61K 9/00

(52) U.S. Cl. 424/47

(57) ABSTRACT

The product for hair treatment includes a pressurized glass container or polyethylene terephthalate container, a sprayable liquid composition in the container, at least one propellant in the container with the composition and a spraying device for spraying the composition contained in the container with the help of the propellant. The composition includes at least one hair-fixing polymer, at least one C2- to C3-univalent alcohol and at least 5 percent by weight water. The propellant is propane, n-butane, isopropane and/or a fluorohydrocarbon. Only a single clear liquid phase is present in the container besides the gas phase.

### BACKGROUND OF THE INVENTION

The subject matter of the invention is a product for hair treatment comprising a pressurized container made of polyethylene terephthalate or glass, a device for spraying a composition present in the container and a sprayable liquid composition comprising a hair-fixing polymers, selected alcohols, at least five percent by weight water and a selected propellant, in which only a single clear liquid phase is present in the container besides a gas phase.

Spray products for hair treatment can be divided into hairsprays and spray lotions. Hairsprays are usually applied as aerosols. They are products, which are sprayed on the hair as finished products for fixing of a set hairstyle after forming or shaping the hair in the hairstyle. In contrast lotions or spray lotions prepare the hair for a subsequent hair styling and are applied to the hair prior to shaping or forming the hair into the hairstyle. The mechanism of action of hair sprays and hair lotions is different. During application of hair sprays individual sprayed drops run on the hairs, collect at the crossing points of individual hair strands and glue them together by depositing fixing polymers after evaporation of the solvent. With lotions the polymer solution applied to the hair spreads on the hair and forms a polymer film at least partially enclosing individual hair and a plurality of individual hairs after evaporation of the solvent.

### SUMMARY OF THE INVENTION

It is an object of the present invention to provide a hair treatment product, which comprises a composition with application properties of a lotion, but can be sprayed like an aerosol hair spray. At the same time the composition should be contained in an economical, non-corrodible, light plastic container, which is transparent if possible. The composition is sufficiently stable cold and is in an attractive, single phase and clear form. The composition requires a minimum amount of water to guarantee the required spreading properties and spray profile. Usually a combination of water-containing compositions with liquid hydrophobic propellant gas is unsuitable, in order to form clear products, since usually an inhomogeneous, uncosmetic and unattractively appearing region having streaking or schlieren forms because of interactions between propellant, effective and auxiliary ingredients and solvents. Turbidity occurs in one or both phases because of the action of the usual ingredients. However transparent containers are desirable because of the ease with which the filling level can be observed in the container and because of an increased attractiveness of the clear transparent products. Replacement of the hydrophobic propellant by hydrophilic propellant, such as dimethyl ether, is problematic, because it is incompatible with the plastic packaging material.

It has now been found that the above-identified object and others, which will be made more apparent hereinafter, are attained by a product for hair treatment comprising:

(A) a pressure-tight glass or polyethylene terephthalate container containing a sprayable liquid composition;

(B) a spraying device for spraying the sprayable liquid composition in the container;

(C) the sprayable liquid composition in the container, which includes

(c1) at least one hair-fixing polymer,

(c2) at least one univalent alcohol selected from the group consisting of ethanol, n-propanol and isopropanol, and

(c3) at least 5, preferably at least 10 percent by weight, water, and

(D) at least one propellant selected from the group consisting of propane, n-butane, isopropane, fluorohydrocarbons and mixtures of the foregoing;

wherein only a single clear liquid phase is present in the container besides the gas phase.

Water is advantageously contained in the liquid composition (C) of the product of the invention, for example, in an amount of 5 to 50, especially of 10 to 40, percent by weight. The amount ratios of liquid composition (C) to propellant (D) amount to, for example, 96:4 to 60:40, especially 93:7 to 70:30. The liquid phase is clear in the sense of the invention, when no turbidity or schlieren or streaking is detectable with the naked eye. A good spray is produced when the average spray droplet size, expressed as dv(50) value, is less than 100 μm, preferably less than 90 μm, or expressed as dv(90) value, is a maximum 160 μm, preferably a maximum 150 μm, when sprayed with a spray head DPV Cosmos with whirl ring 0.020 and DPV valve cone 1×0.010 nylon of DPV. The dv(50) and/or dv(90) values give the respective maximum diameters of 50% and of 90% of all droplets. The droplet size distribution, for example, can be determined with the help of a particle-measuring unit based on light beam deflection, e.g. a Malvern Particle Size Measurement Unit. The spray produced with the product according to the invention preferably has a dv(50) value in a range of 50 to 100 μm, especially of 70 to 90 μm, and a dv(90) value in a range from 90 to 160 μm, especially from 115 to 150 μm.

The subject matter of the present invention is also outstandingly suited to make a preparation with reduced amounts of easily volatilized or volatile organic ingredients (VOC), e.g. VOC 80% or VOC 55%. The product according to the invention thus contains preferably at most 80 percent by weight, especially 55% by weight, of ingredients classified as VOC, based on the total amount of the preparation containing the liquid composition and propellant. The California Air Resources Board (CARB) defines VOC ingredients as substances with a vapor pressure greater than 0.1 mm Hg at 20° C. or as substances with 12 or less carbon atoms, with the exception of certain particular compounds with reduced or no photochemical ozone formation potential (POCP), such as carbon dioxide, methylene chloride, acetone, methyl acetate, FCKWs and fluorinated hydrocarbons.

Hair-fixing Polymers

The hair-fixing polymer (c1) is contained in the composition (C) of the product of the invention in an amount of from 1 to 15 percent by weight, especially preferably of 2 to 8 percent by weight. “Hair-fixing polymers” are understood to be those polymers, which, when used in an amount of from 0.01 to 5% in an aqueous, alcoholic or aqueous-alcoholic solution, are in a position to deposit a polymer film on the hair and thus to fix the hair.

The hair-fixing polymer can be nonionic, anionic, cationic, zwitterionic or amphoteric, in so far as they have sufficient solubility in the solvent and propellant mixture according to the invention, which comprises water, univalent alcohol and hydrophobic propellant. Sufficient solubility means, for example, when at least one percent of the polymer is dissolved. The polymers can be of synthetic or natural origin.

Suitable nonionic polymers are, for example, homopolymers of vinyl caprolactam, vinyl pyrrolidone and N-vinylformamide. Additional suitable synthetic nonionic film-forming hair-fixing polymers are e.g. copolymerizates of vinyl pyrrolidone and vinyl acetate, terpolymers of vinyl pyrrolidone, vinyl acetate and vinyl propionate, polyacrylamides, polyvinyl alcohols and polyethylene glycol/polypropylene glycol copolymers. Suitable polymers of natural origin include e.g. polysaccharides, chitosan, chitosan derivatives, Chinese pine resin, guar and/or guar derivatives, celluose derivatives and shellack. The derivatives of the aforementioned polymers are especially hydroxyalkyl ethers with 1 to 4, preferably 2 to 3, carbon atoms in the alkyl groups, e.g. hydroxyethyl or hydroxyproypl guar or hydroxyethyl or hydroxypropyl cellulose.

Suitable hair-fixing polymers include those containing acid groups. These acid groups are preferably used in unneutralized, i.e. in not ionized, form. The acid groups may include —COOH, —SO₃H, —OSO₃H, —OPO₂H and —OPO₃H groups. Carboxylic acid groups are especially preferred. The polymers with acid groups are e.g. copolymers of at least one first radically polymerizable monomer, which is not ionizable, i.e. has neither acid groups nor amine groups, and a second radically polymerizable monomer, which has acid groups. Radically polymerizable but nonionic monomers include e.g. acryl amide,methacrylamides, alkyl and dialkylacrylamides, alkyl and dialkylmethacrylamides, alkylacrylates, alkylmethacrylates, vinyl caprolactone, vinyl pyrrolidone, vinyl esters, vinyl alcohol, in which the alkyl groups of these monomers preferably contain one to seven carbon atoms, especially preferably from one to three carbon atoms. Radically polymerizable monomers with acid groups are e.g. acrylic acid, methacrylic acid, crotonic acid, itaconic acid, maleic acid and/or maleic acid anhydride or its monoester, fumaric acid or its monoester, aldehydocarboxylic acids or ketocarboxylic acids.

Copolymers made from nonionic monomers and monomers containing acid groups include e.g. especially copolymers of acrylic acid or methacrylic acid with monomers selected from the group consisting of acrylic acid or methacrylic acid esters, acryl amides, methacrylamides and vinylpyrrolidones, copolymers of crotonic acid with monomers selected from the group consisting of vinyl esters, acrylic acid or methacrylic acid esters, acrylamides and methacrylamides, especially vinyl acetate/crotonic acid copolymers (INCI name: VA/crotonates copolymer), vinyl acetate/crotonic acid/polyethylene oxide copolymers, partially esterified copolymers between vinyl methyl ether and maleic acid anhydride, e.g. methylvinylether/maleic acid monoethyl- or -monobutyl ester copolymer (INCI name: ethyl ester of PVM/MA copolymer, butyl ester of PVM/MA copolymer), copolymers of acrylic acid, alkyl acrylate and N-alkylacrylamide (INCI name: acrylates/acrylamide copolymer), especially acrylic acid/ethyl acrylate/N-t-butylacrylamide terpolymer, copolymers of one or more C1-C5-alkylacrylates, especially C2-C4-alkylacrylates and acrylic acid or methacrylic acid (INCI-name: acrylates copolymer), especially methacrylic acid/ethyl acrylate/butylacrylate terpolymer, terpolymers of vinyl acetate/crotonate/alkanoate, especially vinyl acetate/crotonate/vinyl neodecanoate copolymers or vinyl acetate/crotonate/vinyl propionate copolymers (INCI name: VA/crotonates/vinyl neodecanoate copolymer and/or VA/crotonates/vinyl propionates copolymer).

The hair-fixing polymers preferred for the product according to the invention are selected from vinyl ester/crotonic acid copolymers. Vinyl acetate/crotonic acid coplymers, which are used in nonionic, unneutralized form, are especially preferred. The acid number is e.g. in the range of 15 to 60, especially of 30-40 mg KOH/g. A suitable commercial product is for example ARISTOFLEX® A 60.

Univalent Alcohols

The univalent alcohol (c2) is contained in the liquid composition (C) of the product according to the invention, for example, in an amount of 45 to 90, especially 55 to 85, percent by weight. It can be ethanol, isopropanol, n-propanol, mixtures of ethanol and isopropanol, of ethanol and n-propanol, of isopropanol and n-propanol, or a mixture of ethanol, isopropanol and n-propanol. Ethanol, isopropanol or a mixture of ethanol and isopropanol are preferred as the univalent alcohol. The liquid composition can contain additional solvents, in so far as the clarity of the composition is not impaired. The additional solvents can be univalent alcohols, such as methanol, butanol or pentanol; or multivalent alcohols with 2 to 6 carbon atoms.

Propellant

The propellant according to the invention is a hydrophobic substance, which under standard conditions, i.e. standard pressure 1013 mbar and room temperature (20° C.), is present in the container in liquid or dissolved form after filling. Especially C₃- to C₄-hydrocarbons, such as propane, n-butane, isobutene, fluorohydrocarbon materials or mixtures of the foregoing, are suitable. Fluorohydrocarbons include e.g. F152a (1,1-difluoroethane) or F 134 (tetrafluoroethane). Mixtures of propane and n-butane, propane and isobutene, as well as a mixture of propane, n-butane and isobutene, are especially preferred. The content of univalent alcohols in the liquid composition is preferably greater than or equal to 70 percent by weight and the content of water is preferably less than or equal to 25 percent by weight in the case of the hydrocarbon propellants. In the case of the fluorohydrocarbons the content of univalent alcohols in the liquid composition is preferably greater than or equal to 60 percent by weight and the content of water is preferably less than or equal to 35 percent by weight. The type and amount of the propellant is preferably selected so that the pressure in the container is from e.g. 1 to 3, especially from 2 to 2.7, bar at 20° C.

Container

The product according to the invention is packed in a suitable pressure-tight aerosol container and has a device, which permits spraying of the liquid composition by means of the propellant, as an additional component. For example, a commercial aerosol spray head can be used as a suitable spraying device. The container is made from polyethylene terephthalate and is preferably transparent, so that the consistency, the filling state height, the color of the composition of the liquid phase can be observed from the outside of the container. Another transparent material is e.g. glass.

Additional Additive Ingredients

The liquid compositions according to the invention can also contain additional conventional additive ingredients for hair treatment agents, in so far as the clarity of the product is not impaired. These conventional additive ingredients include e.g. preservatives, emulsifiers, solvating agents, perfume oils, fragrances, pH buffer substances, care ingredients, such as plant and vegetable extracts, protein hydroyzates, silk hydrolyzates, silicone compounds, light-protective agents, anti-oxidants, radical trapping agents, anti-flaking agents, luster-imparting substances, vitamins, softeners, compatibility improving substances, coloring agents, etc.

Preferred Embodiments

The following products according to the invention are especially preferred.

A preferred product according to the invention in which the liquid composition contains

(c1) from 1 to 8 percent by weight of a non-neutralized vinyl acetate/crotonic acid copolymer;

(c2) from 70 to less than 90 percent by weight of at least one univalent alcohol, selected from ethanol, n-propanol and isopropanol; and

(c3) from greater than 10 to 25 percent by weight, water; and in which the propellant is selected from propane, n-butane, isobutane and their mixtures, and wherein an amount ratio of liquid composition to propellant amounts to from 93:7 to 70:30 and the pressure in the container amounts to at maximum 3 bar at 20° C.

An additional preferred product according to the invention in which the liquid composition contains

(c1) from 1 to 8 percent by weight of a non-neutralized vinyl acetate/crotonic acid copolymer;

(c2) from 60 to less than 90 percent by weight of at least one univalent alcohol, selected from ethanol, n-propanol and isopropanol; and

(c3) from greater than 10 to 35 percent by weight, water; and in which the propellant is selected from propane, n-butane, isobutene and their mixtures, and wherein an amount ratio of liquid composition to propellant amounts to from 93:7 to 70:30 and the pressure in the container amounts to at maximum 3 bar at 20° C.

The subject matter of the invention is also a method of hair treatment according to the invention, in which the liquid composition is applied to the hair by means of the product according to the invention and subsequently a hairstyle or hair-do is set up.

The cosmetic product according to the invention is used, in which an amount of the composition (typically about 1 to 10 g, especially 2 to 7 g) sufficient for obtaining the desired hair-fixing effect according to the abundance and state of the hair is sprayed on the hair. The desired hairstyle or hair-do is set up and the hair is dried as necessary. Subsequently a conventional hairspray can be used as finish for fixing the set-up hairstyle or hair-do.

The product can be used on moist and dry hair. It can be used as a styling lotion and applied at home or in a beauty parlor. The product can be sprayed on the dried, already set-up hair and then dried or serve for refreshing the hairstyle or hair-do.

### EXAMPLES

### Example 1

### Aerosol Lotion

| | | |
|---|---|---|
| 5 | g | ARISTOFLEX ® A60¹⁾ |
| 80 | g | Ethanol |
| 0.2 | g | Perfume |
| to 100 | g | Water |

¹⁾60 percent solution of vinyl acetate/crotonic acid copolymer in 35% isopropanol and 5% water

The liquid active solution is filled into a pressure-tight transparent polyethylene terephthalate or glass container together with a propane/butane mixture (2.7 bar) with an amount ratio of 90:10 and provided with an aerosol spray head.

### Example 2

### Aerosol Lotion (80 % VOC)

| | | |
|---|---|---|
| 5.6 | g | ARISTOFLEX ® A60¹⁾ |
| 75.6 | g | Ethanol |
| 0.2 | g | Perfume |
| to 100 | g | Water |

¹⁾60 percent solution of vinyl acetate/crotonic acid copolymer in 35% isopropanol and 5% water

The liquid active solution is filled into a pressure-tight transparent polyethylene terephthalate or glass container together with a propane/butane mixture (2.7 bar) with an amount ratio of 90:10 and provided with an aerosol spray head.

### Example 3

### Aerosol Lotion (55 % VOC)

| | | |
|---|---|---|
| 6 | g | ARISTOFLEX ® A60¹⁾ |
| 66 | g | Ethanol |
| 0.2 | g | Perfume |
| to 100 | g | Water |

¹⁾60 percent solution of vinyl acetate/crotonic acid copolymer in 35% isopropanol and 5% water

The liquid active solution is filled into a pressure-tight transparent polyethylene terephthalate or glass container together with a fluorohydrocarbon 152A (1,1-difluoroethane) with an amount ratio of 80:20 and provided with an aerosol spray head.

The disclosure in German Patent Application 102 57 858.3 of Dec. 11, 2002 is incorporated here by reference. This German Patent Application describes the invention described hereinabove and claimed in the claims appended hereinbelow and provides the basis for a claim of priority for the instant invention under 35 U.S.C. 119.

While the invention has been illustrated and described as embodied in a clear hair lotion sprayable as an aerosol, it is not intended to be limited to the details shown, since various modifications and changes may be made without departing in any way from the spirit of the present invention.

Without further analysis, the foregoing will so fully reveal the gist of the present invention that others can, by applying current knowledge, readily adapt it for various applications without omitting features that, from the standpoint of prior art, fairly constitute essential characteristics of the generic or specific aspects of this invention.

What is claimed is new and is set forth in the following appended claims.

We claim:

1. A product for hair treatment comprising:
(A) a pressure-tight glass or polyethylene terephthalate container containing a sprayable liquid composition;
(B) a spraying device for spraying the sprayable liquid composition in the container;
(C) said sprayable liquid composition contained in the container, said sprayable liquid composition containing
(c1) at least one hair-fixing polymer,
(c2) at least one univalent alcohol selected from the group consisting of ethanol, n-propanol and isopropanol, and
(c3) at least five percent by weight of water; and
(D) at least one propellant selected from the group consisting of propane, n-butane, isopropane and fluorohydrocarbons;
wherein only a single clear liquid phase is present in the container besides a gas phase.

2. The product as defined in claim 1, wherein said at least one hair-fixing polymer has acid groups.

3. The product as defined in claim 1, wherein said at least one hair-fixing polymer is a copolymer of at least one first radically polymerizable nonionic monomer and a second radically polymerizable monomer containing acid groups.

4. The product as defined in claim 3, wherein said at least one hair-fixing polymer is a non-neutralized vinyl acetate/crotonic acid copolymer.

5. The product as defined in claim 1, wherein said sprayable liquid composition contains from 1 to 15 percent by weight of said at least one hair-fixing polymer.

6. The product as defined in claim 1, wherein said sprayable liquid composition contains from 5 to 50 percent by weight of said water and from 45 to 90 percent by weight of said at least one univalent alcohol.

7. The product as defined in claim 1, wherein an amount ratio of said sprayable liquid composition to said propellant is from 96:4 to 60:40.

8. The product as defined in claim 1, wherein said container is pressurized up to a maximum pressure of 3 bar at 20° C.

9. The product as defined in claim 1, wherein a sum of all volatile organic ingredients present in said container is not more than 80 percent by weight based on a total amount of said propellant and said composition.

10. The product as defined in claim 1, wherein said sprayable liquid composition contains from 1 to 8 percent by weight of a non-neutralized vinyl acetate/crotonic acid copolymer as said at least one hair-fixing polymer, from 70 to 90 percent by weight of said at least one univalent alcohol and from greater than 10 to 25 percent by weight of said water, and wherein said at least one propellant is selected from the group consisting of said propane, said n-butane and said isopropane and an amount ratio of said sprayable liquid composition to said propellant is from 93:7 to 70:30 and said container is pressurized up to a maximum pressure of 3 bar at 20° C.

11. The product as defined in claim 1, wherein said sprayable liquid composition contains from 1 to 8 percent by weight of a non-neutralized vinyl acetate/crotonic acid copolymer as said at least one hair-fixing polymer, from 60 to 90 percent by weight of said at least one univalent alcohol and from greater than 10 to 35 percent by weight of said water, and wherein said propellant is said fluorohydrocarbon, and an amount ratio of said sprayable liquid composition to said propellant is from 93:7 to 70:30 and said container is pressurized up to a maximum pressure of 3 bar at 20° C.

12. A method for treating hair, said method comprising the steps of:
a) applying the sprayable liquid composition of the product of claim 1 to the hair; and subsequently
b) setting the hair in a hairstyle or hairdo.
